(19) United States
(12) Patent Application Publication
(10) Pub. No.: US 2015/0283382 A1
(43) Pub. Date: Oct. 8, 2015

(54) Respiration Sensors For Recording Of Triggered Respiratory Signals In Neurostimulators

(71) Applicant: MED-EL Elektromedizinische Geraete GmbH, Innsbruck (AT)

(72) Inventors: Christian Denk, Innsbruck (AT); Markus Oberparleiter, Rum (AT)

(21) Appl. No.: 14/677,023

(22) Filed: Apr. 2, 2015

Related U.S. Application Data
(60) Provisional application No. 61/975,071, filed on Apr. 4, 2014.

(51) Int. Cl. A61N 1/36

(52) U.S. Cl. CPC A61N 1/36135; A61N 1/3601; A61N 1/3611

(57) ABSTRACT

A respiration implant system includes a parasternal respiration sensor is configured for intramuscular placement into parasternal muscle of the implanted patient to develop a respiration signal representing respiration activity of the implanted patient. A movement sensor develops a patient movement signal representing movement of the implanted patient. A pacing processor receives the respiration signal from the respiration sensor and the movement signal from the movement sensor to generate a respiration pacing signal synchronized with the respiration activity of the implanted patient. A stimulating electrode delivers the respiration pacing signal from the pacing processor to respiration neural tissue of the implanted patient to promote the breathing effort of the implanted patient.

This application claims priority from U.S. Provisional Patent Application 61/975,071, filed Apr. 4, 2014, which is incorporated herein by reference in its entirety.

### TECHNICAL FIELD

The present invention relates to respiration implant systems such as implantable respiration pacing systems and sleep apnea treatment systems.

### BACKGROUND ART

The larynx is located in the neck and is involved in breathing, producing sound (speech), and protecting the trachea from aspiration of food and water. FIG. 1A shows a coronal section view and FIG. 1B shows a transverse section view of the anatomy of a human larynx including the epiglottis 101, thyroid cartilage 102, vocal folds 103, cricothyroid muscle 104, arytenoid cartilage 105, posterior cricoarytenoid muscle (PCAM) 106, vocalis muscle 107, cricoid cartilage 108, recurrent laryngeal nerve (RLN) 109, transverse arytenoid muscle 110, oblique arytenoid muscle 111, superior laryngeal nerve 112, and hyoid bone 113.

The nerves and muscles of the larynx abduct (open) the vocal folds 103 during the inspiration phase of breathing to allow air to enter the lungs. And the nerves and muscles of the larynx adduct (close) the vocal folds 103 during the expiration phase of breathing to produce voiced sound. At rest, respiration frequency typically varies from 12 to 25 breaths per minute. So, for example, 20 breaths per minute result in a 3 second breath duration, with 1.5 sec inspiration, and 1.5 sec exhalation phase (assuming a 50/50 ratio). The breathing frequency changes depending on the physical activity.

Unilateral and bilateral injuries or ruptures of the recurrent laryngeal nerve (RLN) 109 initially result in a temporal partial paralysis of the supported muscles in the larynx (and the hypolarynx). A bilateral disruption of the RLN 109 causes a loss of the abductor function of both posterior cricoarytenoid muscles (PCAM) 106 with acute asphyxia and life-threatening conditions. This serious situation usually requires surgical treatment of the bilateral vocal cord paralysis such as cordotomy or arytenoidectomy, which subsequently restrict the voice and puts at risk the physiologic airway protection.

Another more recent treatment approach to RLN injuries uses a respiration implant that electrically stimulates (paces) the PCAM 106 during inspiration to abduct (open) the vocal folds 103. During expiration, the vocal folds 103 relax (close) to facilitate voicing. In first generation respiration implant systems, the patient can vary the pacing/respiration frequency (breaths per minute) according to his physical load (at rest, normal walking, stairs, etc.) by manually switching the stimulation frequency of the pacer device, the assumption being that the human body may adapt to the artificial externally applied respiration frequency—within some locking-range. Thus the patient and the respiration pacemaker can be described as free running oscillators at almost the same frequency, but without phase-matching (no phase-locking). Sometimes both systems will be in phase, but other times the systems will be out of phase and thus the benefit for the patient will be reduced.

More recent second generation respiration implants generate a stimulation trigger signal to synchronize the timing of stimulation of the pacemaker to the respiration cycle of the patient. The stimulation trigger signal defines a specific time point during the respiration cycle to initiate stimulation of the target neural tissue. The time point may specifically be the start or end of the inspiratory or expiratory phase of breathing, a breathing pause, or any other defined time point. To detect the desired time point, several types of respiration sensors have been investigated to generate a respiration sensing signal that varies within each breathing cycle. These include, for example, various microphones, accelerometer sensors, and pressure sensors (positioned in the pleura gap). Electromyogram (EMG) measurements also are under investigation for use in developing a stimulation trigger signal.

Besides laryngeal pacemakers for RLN injuries, there also are respiration implant neurostimulators that electrically stimulate the hypoglossal nerve that innervates the root of the tongue for treatment of sleep apnea. These sleep apnea treatment systems use a respiration sensor that is implemented to trigger on the inhaling phase of breathing, for example, using a bioimpedance measurement or a pressure sensor in the pleural gap.

### SUMMARY

Embodiments of the present invention are directed to respiration implant systems (e.g., laryngeal pacemaker systems and sleep apnea treatment systems) for an implanted patient with impaired breathing. A parasternal respiration sensor is configured for intramuscular placement into a parasternal muscle of the implanted patient to develop a respiration signal representing the respiration cycle of the implanted patient. A movement sensor is configured to develop a patient movement signal representing movement of the implanted patient. A pacing processor is configured to receive the respiration signal from the respiration sensor and the movement signal from the movement sensor to generate a respiration pacing signal synchronized with the respiration activity of the implanted patient. A stimulating electrode is configured to deliver the respiration pacing signal from the pacing processor to respiration neural tissue of the implanted patient to promote the breathing effort of the implanted patient.

In further specific embodiments, the movement sensor may be a three-axis accelerometer and/or a gyroscope. The respiration sensor may be configured to detect a breathing pause or the onset or end of inspiratory respiration activity in the implanted patient. The respiration sensor may be an intramuscular pressure sensor or an electromyographic (EMG) sensor. In the latter case, the pacing processor may be further configured to extract in addition to the EMG activity, an electrocardiogram (ECG) signal for generating the respiration pacing signal.

The respiration implant system may be a laryngeal implant system and the stimulating electrode delivers the respiration pacing signal to the posterior cricoarytenoid muscle in the larynx. Or the respiration implant system may be a sleep apnea treatment system and the stimulating electrode delivers the respiration pacing signal to the hypoglossal nerve or the iSLN (internal superior laryngeal nerve).

### BRIEF DESCRIPTION OF THE DRAWINGS

FIG. 1A shows a coronal section view and FIG. 1B shows a transverse section view of the anatomy of a human larynx.

FIG. 2 shows a respiration implant system according to an embodiment of the present invention.

FIG. 3 shows waveforms for the pressure change and respiratory cycle for a parasternal intramuscular pressure catheter respiration sensor compared to a reference respiratory signal of a thoracic belt measuring the respiratory cycle of expansion of the thorax.

FIG. 4 compares a spirometer reference signal waveform to a parasternal EMG respiration sensor waveform during normal breathing.

FIG. 5 shows waveforms related to electrocardiogram derived respiration (EDR).

FIG. 6 shows an EMG signal and ECG signal from a recording electrode in the parasternal muscle in a respiration implant system.

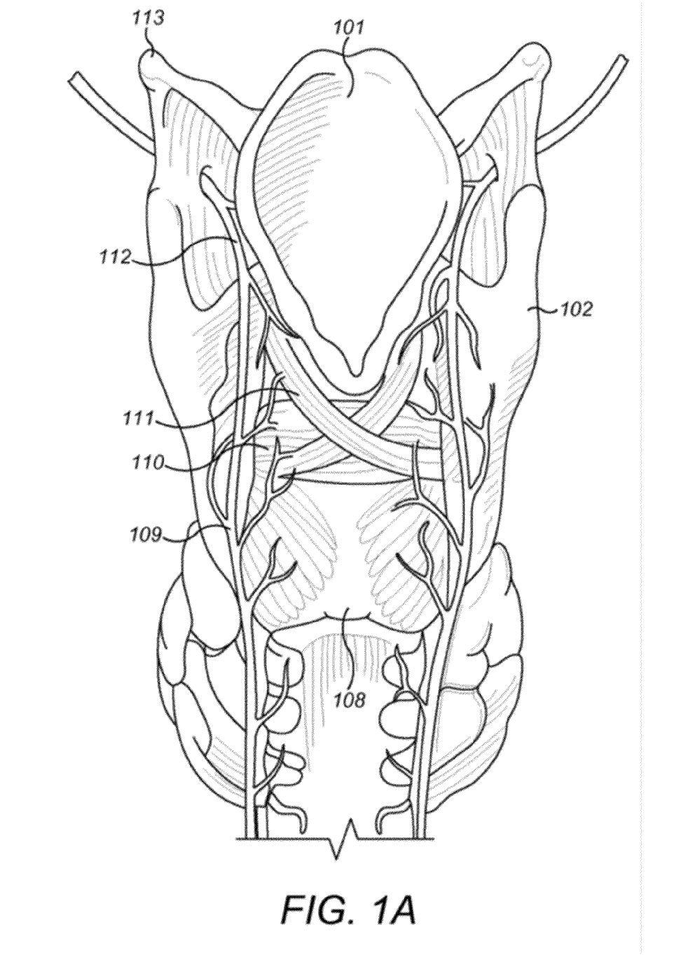

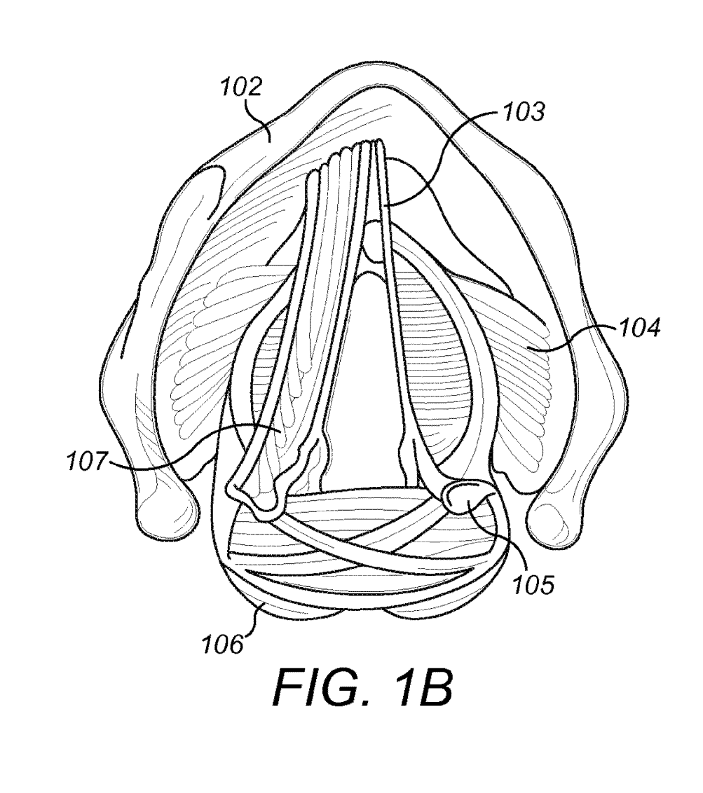

### DETAILED DESCRIPTION

Various embodiments of the present invention are directed to improved respiration implants that use a respiration sensor implanted in the parasternal muscle to detect respiration activity in the patient with impaired breathing, together with a three-axis acceleration sensor and/or a gyroscope as a movement/position sensor. These sensors may be in the specific form of a small device package located inside the main implant housing or outside in a separate housing communicatively connected to the main implant housing. Such respiration implant systems include, for example, laryngeal pacemaker systems and sleep apnea treatment systems.

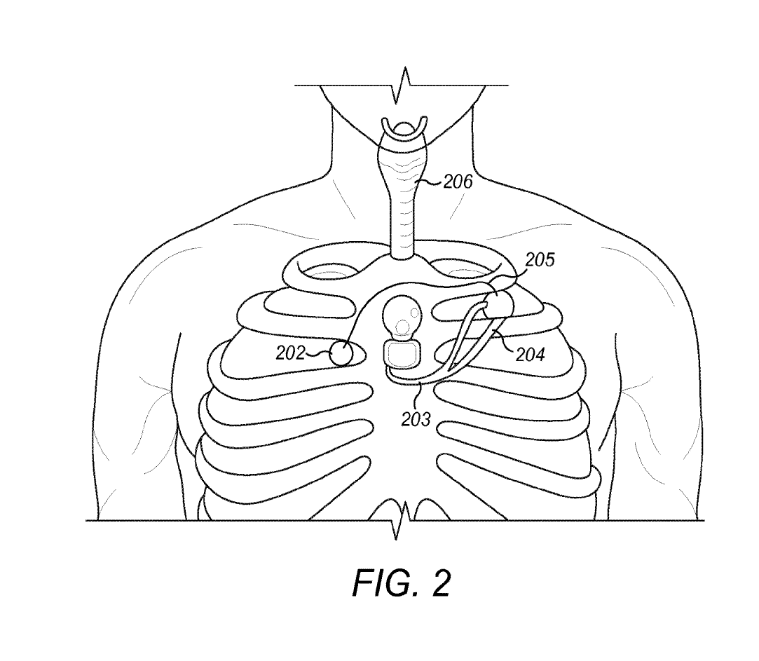

FIG. 2 shows one embodiment of such a respiration implant system with an implanted pacing processor 201 that receives a respiration signal from an implanted respiration sensor 202 implanted in the parasternal muscle that detects respiration activity in the implanted patient. Optionally, a three-axis acceleration movement sensor is located within the housing of the pacing processor 201 and generates a movement signal. Based on the respiration signal, the pacing processor 201 generates a respiration pacing signal that is synchronized with the detected respiration activity and delivers the pacing signal via a processor lead 203 and lead interface 204 to a stimulating electrode 206 implanted in the target respiration neural tissue to promote breathing of the implanted patient.

With respect to the specific implementation of a respiration sensor 202, it will be appreciated that during the inspiration phase of breathing, the various respiratory muscles—such as the diaphragm, intercostal externi, and the parasternal part of the intercostal interni muscles (the latter in the following being referred to as the parasternal muscle)—are always active. With every breathing cycle, during inspiration those muscles are involuntarily active and contract. In particular, the parasternal muscle (also called the intercartilaginous muscle or the parasternal part of the intercostal interni muscle) elevates the ribs during inspiration. Thus, some specific embodiments of the present invention use the contraction of the parasternal muscle, specifically via a parasternal respiration sensor 202 implemented as, for example, an electromyogram (EMG) sensor, pressure sensor.

The parasternal muscle has a medial to dorsal gradient of activity and muscle mass as well as a cranial caudal gradient, which means that the parasternal muscle becomes smaller from the sternum to the base of the rib. A parasternal respiration sensor 202 can be inserted into the parasternal muscle in a rib interspace in the thorax near the sternum and the pacing processor (see FIG. 2), preferably in the 2nd or 3^{rd} interspace, and a reliable respiration signal from the parasternal muscle is thereby available. The 2nd or 3rd interspace provides the thickest proportion of the muscle (around 6-10 mm) in which to secure the respiration sensor 202, and also provides the smallest part of the pectoralis muscle where there is the least overshadowing effect or cross-contamination to be expected. In addition, this location is spatially close to the pacing processor 201 and the surgical placement of the respiration sensor 202 into the parasternal muscle is minimally invasive and surgically uncomplicated.

A parasternal respiration sensor 202 in combination together with a movement sensor/gyroscope also can avoid mis-stimulation during active rotation and bending of the thorax. The parasternal muscle is known to be active during rotation and bending of the thorax, and with the help of the movement sensor these specific bendings and rotations can be measured and mis-stimulation avoided.

As mentioned above, the respiration sensor 202 may be in the specific form of a pressure sensor; e.g., a pressure catheter implanted into the parasternal muscle. The resulting pressure signal then depends on the contraction of the parasternal muscle itself, and also is directly influenced by the intra-thoracic pressure. Therefore there will be always a combination of intramuscular pressure and intra-thoracic pressure measured. Other prior arrangements have placed a pressure catheter into the pleural gap or space rather than intramuscularly as here.

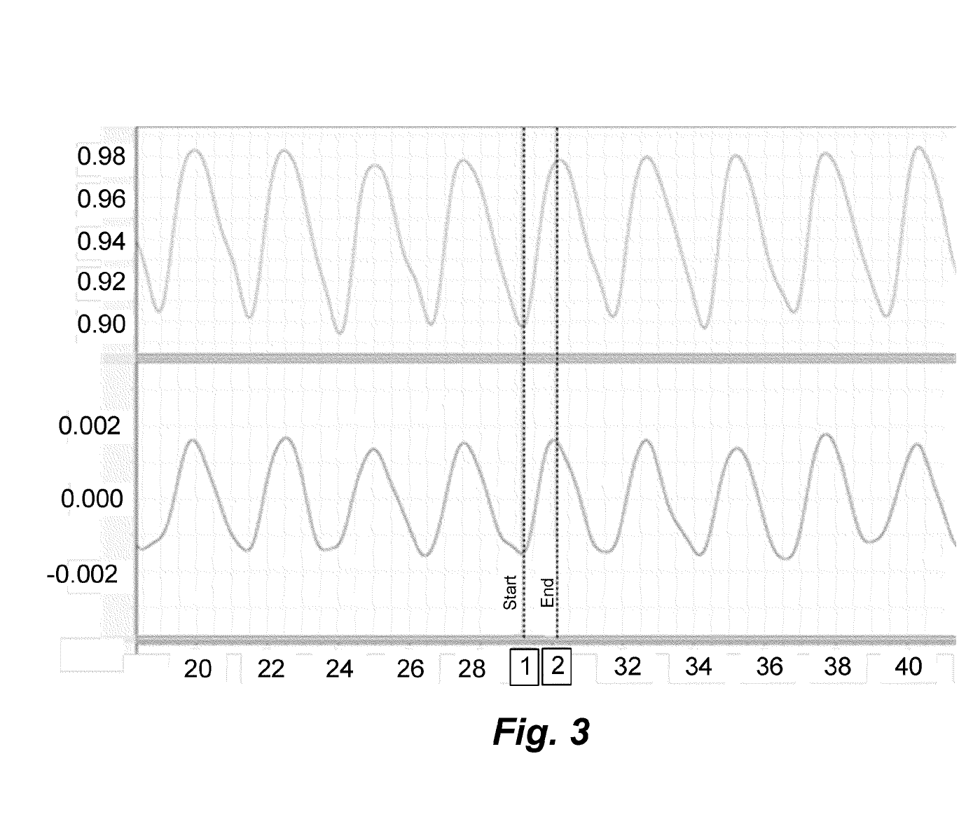

A typical waveform for the pressure change and respiratory cycle for a parasternal intramuscular pressure catheter respiration sensor is shown in the top part of FIG. 3, and a comparable reference respiratory signal of a thoracic belt measuring the respiratory cycle of expansion of the thorax is shown in the bottom part of FIG. 3. Each waveform minimum reflects the starting of inspiration as shown by the left-hand vertical dashed line, and waveform maximum indicates the end of inspiration and the start of expiration as shown by the right-hand vertical dashed line. The pacing processor 201 can use signal processing of the respiration signal from the parasternal pressure sensor 202 to detect the onset of inspiration.

A pressure catheter respiration sensor device with a diameter from 1-2 mm is thin enough to be placed into the parasternal muscle (around 6-10 mm), so that involuntary muscle contractions from the parasternal muscle then provide a reliable breathing signal in different circumstances.

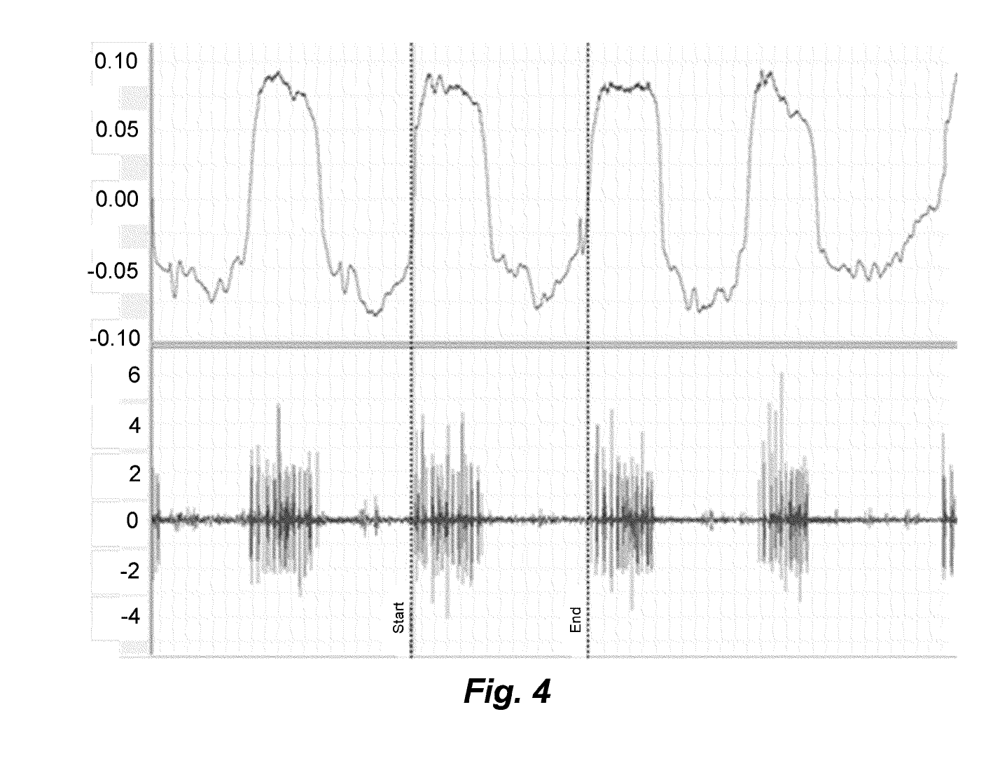

Rather than a pressure sensor, the elevation of the ribs by the inspiratory intercostal muscles during inspiration also can be measured via electromyography (EMG) with an intramuscular EMG respiration sensor 202 that senses the electrical activity of the parasternal muscle and detect the onset of inspiration. The placement of a parasternal EMG respiration sensor 202 may be as discussed above for a parasternal pressure sensor, in the parasternal muscle close to the sternum to avoid overshadowing EMG activity of the sensed EMG signal. FIG. 4 compares a spirometer reference signal waveform in the upper portion where positive signals are inspiratory and negative are expiratory, to a parasternal EMG respiration sensor waveform shown in the lower portion. Because the stimulating electrode 206 is placed into the PCA muscle in the larynx relatively distant from the parasternal muscle, no stimulation artifacts will be visible in the parasternal EMG respiration signal.

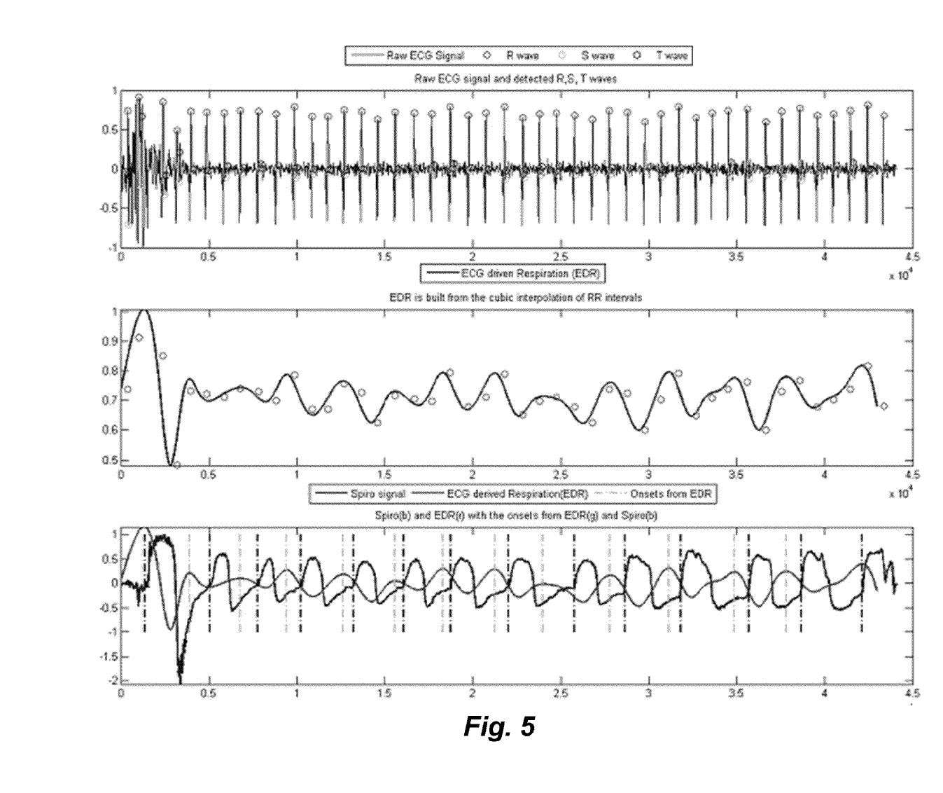

An EMG respiration sensor will also detect the largest bio signal in the human body, the electrocardiogram (ECG). Expansion and deflation of the lung during respiration will also move the heart, and the electrical axis of the heart also moves during respiration. This suggests that the EMG and ECG signals might be used together to generate a respiration trigger signal; for example, by detecting the onset of inspiration. The R-wave is the largest signal in the ECG and the easiest to detect, as shown in the top waveform in FIG. 5. The R-wave height (RWH) can be taken and a cubic interpolation can be made for each RWH to produce a representation of the respiration, as shown in the middle waveform in FIG. 5, which is referred to as the electrocardiogram-derived respiration (EDR). The bottom waveform in FIG. 5 overlays the EDR with a spirometer reference signal to confirm that the EDR tracks with the respiration cycle of inspiration and expiration.

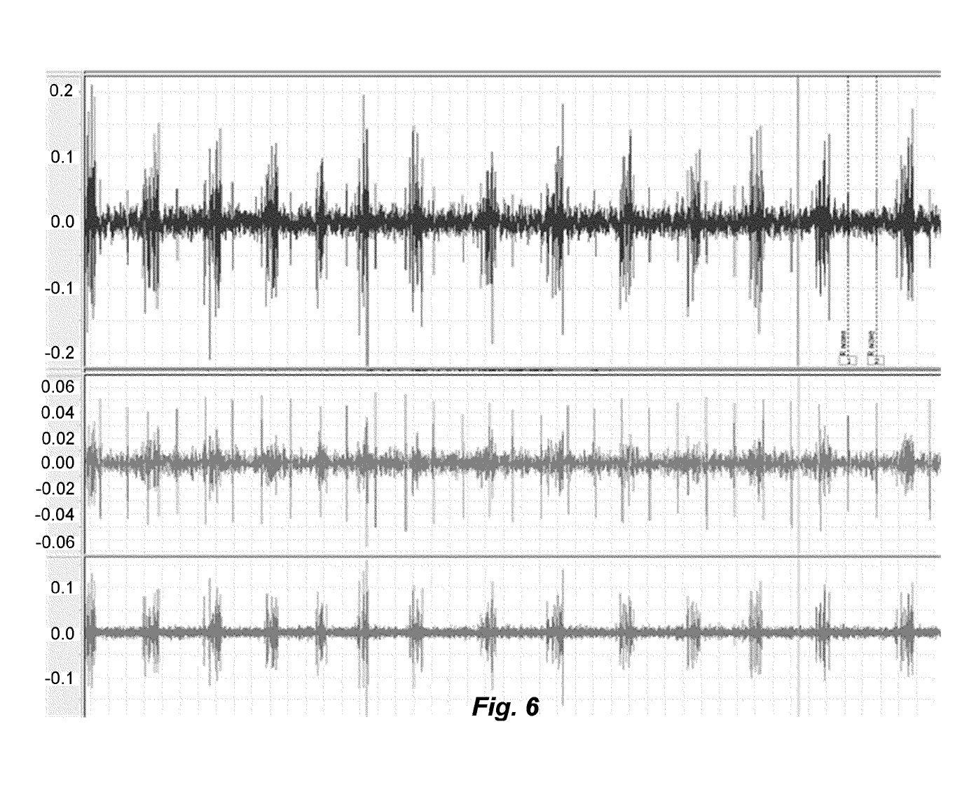

In FIG. 6 the top waveform shows the parasternal EMG overlaid with the R-wave peaks of the ECG showing a typical raw waveform as measured by a parasternal EMG sensor. In the middle waveform in FIG. 6, a band pass filter around 10-60 Hz accentuates the ECG R-wave peaks. In the bottom waveform in FIG. 6, a different band pass filter (50-400 Hz) shows extraction of the EMG signal. For each signal, an inspiratory onset can be calculated and the combination of both signals can be used to generate a trigger stimulus for a respiration implant. The addition of the ECG signal extraction can provide a second set of respiratory sensor within the same recording and add value for the detection of the onset of inspiration.

Besides for treatment of impaired laryngeal structures via stimulation of the posterior cricoarytenoid muscle, embodiments of the present invention also may be useful for treatment of sleep apnea. During sleep for those afflicted with this disease, apnea events occur where the airway is blocked (obstructive apneic event) and no air comes in or goes out. The respiratory effort increases (hypopnea) and increases greatly if the airway becomes blocked (apnea). In that situation, all respiratory muscles including the parasternal muscle show increased activation to restart the airflow.

This increased neural activity can be recorded as an increase in respiratory effort in the parasternal muscle sensor, either based on the pressure difference between inspiration and expiration (maximum to minimum pressure) for each breath, or the increased EMG activity can be detected, for example based on the EMG rectified band pass filtered signal between inspiration and expiration. And in such an apnea treatment system, an acceleration-based movement sensor as discussed above can be useful to automatically detect when a person is lying down and sleeping in order to start measuring the respiration signal from the respiration sensor to detect an apnea event. For example, the band pass filtered acceleration signal from the movement sensor (0.1-0.5 Hz) would indicate movement related acceleration from movement of the rib cage.

So if the change in respiratory effort (as determined from the respiration signal and the movement signal) exceeds a certain threshold level and has been increased over recent breaths, then an apnea event is detected. Then the pacing processor can use the respiration signal from the parasternal respiration sensor to determine a given specific point during the respiratory cycle, for example, the start or end of inspiration or expiration. The pacing processor then generates a respiratory-synchronized stimulus signal to the stimulation site (e.g., the hypoglossal nerve or the iSLN) until an improvement is detected in the respiration signal that indicates that the apnea event has been resolved. In some embodiments, triggered respiratory stimulation may commence as soon as the respiratory effort increases slightly in order to prevent the apnea. In other embodiments an untriggered and continuous stimulation may commence as soon as the respiratory effort increases slightly in order to prevent a hypopnea or obstruction during the whole sleep phase.

Embodiments of the invention may be implemented in part in any conventional computer programming language such as VHDL, SystemC, Verilog, ASM, etc. Alternative embodiments of the invention may be implemented as pre-programmed hardware elements, other related components, or as a combination of hardware and software components.

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A respiration implant system for an implanted patient with impaired breathing, the system comprising:
a parasternal respiration sensor configured for intramuscular placement into parasternal muscle of the implanted patient to develop a respiration signal representing respiration activity of the implanted patient;
a movement sensor configured to develop a patient movement signal representing movement of the implanted patient;
a pacing processor configured to receive the respiration signal from the respiration sensor and the movement signal from the movement sensor to generate a respiration pacing signal synchronized with the respiration activity of the implanted patient; and
a stimulating electrode configured to deliver the respiration pacing signal from the pacing processor to respiration neural tissue of the implanted patient to promote the breathing effort of the implanted patient.

2. The system according to claim 1, wherein the movement sensor is a three-axis accelerometer.

3. The system according to claim 1, wherein the respiration sensor is configured to detect a breathing pause or onset or end of inspiratory respiration activity in the implanted patient.

4. The system according to claim 1, wherein the respiration sensor is an electromyographic (EMG) recording electrode.

5. The system according to claim 1, wherein the pacing processor is further configured to extract an electrocardiogram (ECG) signal from the respiration signal for generating the respiration pacing signal.

6. The system according to claim 1, wherein the respiration sensor is an intramuscular pressure sensor.

7. The system according to claim 1, wherein the respiration implant system is a laryngeal implant system and the stimulating electrode delivers the respiration pacing signal to the posterior cricoarytenoid muscle in the larynx.

8. The system according to claim 1, wherein the respiration implant system is a sleep apnea treatment system and the stimulating electrode delivers the respiration pacing signal to the hypoglossal nerve or the internal superior laryngeal nerve (iSLN).

9. A method of developing a respiration pacing signal in a patient with impaired breathing to promote breathing effort of the implanted patient, the method comprising:
using a respiration sensor implanted into parasternal muscle of the implanted patient to develop a respiration signal representing respiration activity of the implanted patient;
developing a patient movement signal representing movement of the implanted patient;
generating a respiration pacing signal synchronized with the respiration activity of the implanted patient based on the respiration signal and the movement signal; and
delivering the respiration pacing signal to respiration neural tissue of the implanted patient to promote the breathing effort of the implanted patient.

10. The method according to claim 9, wherein the patient movement signal is developed from a three-axis accelerometer.

11. The method according to claim 9, wherein the respiration signal represents onset of inspiratory respiration activity in the implanted patient.

12. The method according to claim 9, wherein the respiration signal is an electromyographic (EMG) signal.

13. The method according to claim 12, wherein generating a respiration pacing signal includes extracting an electrocardiogram (ECG) signal from the EMG signal.

14. The method according to claim 9, wherein the respiration signal is a pressure signal.

15. The method according to claim 9, wherein the respiration pacing signal is delivered by a stimulating electrode to the posterior cricoarytenoid muscle in the larynx of the implanted patient.

16. The method according to claim 9, wherein the respiration pacing signal is delivered by a stimulating electrode to the hypoglossal nerve or the internal superior laryngeal nerve (iSLN) of the implanted patient.
